(19) United States
(12) Patent Application Publication
(10) Pub. No.: US 2024/0335586 A1
(43) Pub. Date: Oct. 10, 2024

(54) ELECTROSPUN MICROFIBROUS POROUS STRETCHABLE MEMBRANES AND THE METHOD OF PREPARATION THEREOF

(71) Applicant: Amrita Vishwa Vidyapeetham, Kochi, Kerala (IN)

(72) Inventors: Binulal Nelson Sathy, Kochi, Kerala (IN); Shantikumar Nair, Kochi, Kerala (IN); Niji Nandakumar, Kochi, Kerala (IN)

(21) Appl. No.: 18/681,443

(22) PCT Filed: Aug. 2, 2022
(86) PCT No.: PCT/IN2022/050692

§ 371 Date: Feb. 5, 2024

(30) Foreign Application Priority Data
Aug. 6, 2021 (IN) 202141035539

(51) Int. Cl. A61L 27/26; A61L 27/22; A61L 27/44; A61L 27/46; A61L 27/56; D01D 5/00

(52) U.S. Cl. CPC A61L 27/26; A61L 27/222; A61L 27/227; A61L 27/446; A61L 27/46; A61L 27/56; D01D 5/0038; A61L 2300/252

(57) ABSTRACT

The present invention discloses a highly stretchable matrix, comprising mesh of lattice structures of microfibrillar filaments, having pore size enlargeable up to 8 times by moving the microfibrillar filaments perpendicular to the longitudinal axis of the microfibrillar filaments without losing its integrity. The invention also pertains to a method of preparing said highly stretchable matrix comprising the steps of: electrostatic spinning of the polymeric solution as microfibers, creating an air-flow at the inter-phase of microfibers to completely eliminate the solvent from the surface of the microfibers bundles to avoid inter-fibrillar bonding after collection and dispersing the microfibers in a direction perpendicular to the longitudinal axis of the fibers, 6-12 times the original width using a dispersion unit to obtain a stretch responsive fibrillar matrix.

### FIELD OF THE INVENTION

The present invention relates to the field of medical devices and applications. The invention pertains to flexible, porous, stretchable polymeric matrix comprising mesh of lattice structures of microfibrillar filaments made from polymeric solution comprising one or more synthetic polymers dissolved in one or more organic volatile solvents. The invention also pertains to process for preparation of said flexible, porous, stretchable polymeric matrix. The polymeric matrix of the invention finds applications as scaffolds to facilitate tissue regeneration or wound healing and closure in vivo or cell isolation and expansion in vitro.

### BACKGROUND ART

Electrospun matrices made out of continuous polymeric fibers with nano-to-micro scale diameters offer high surface-to-volume ratio and interconnected porosity for biomedical applications associated with tissue regeneration and repair compared to polymeric matrices developed through other biofabrication techniques. However, the porosity and pore size available in the electrospun matrices are not adequate to facilitate movement of cells, capillaries and nerves across the thickness of the matrices. [1]. In addition, the static pore sizes present in the currently available electrospun polymeric matrices are not responsive to the traction forces exerted by the cells during tissue formation which can hinder tissue remodeling. Altogether these limitations result in a barrier-like behaviour in electrospun matrices while using as scaffolds for tissue regeneration thus limiting its potential application in the biomedical field.

Electrospun flexible matrices, having pores whose sizes and volumes change dynamically by itself in response to traction or external forces would facilitate accommodation of growing cells to squeeze through said pore walls to get transported within the scaffold. However, such an effect could not be accomplished by electrospun matrices with rigid pore-walls. Thus, the need of the hour in the biomedical field of tissue repair and engineering is to have a polymeric matrix with a pore architecture having the ability to dynamically remodel itself during the regenerative and healing process. Further, the sizes and volumes of the pores in the matrix must be adequate in all three spatial dimensions (x, y and z) to allow for through-the-thickness cell transport for regenerative processes, including tissue regeneration and wound healing. Fibrous matrices having the above features have not been conceived before. While normal woven cloth is a possibility for such applications, these materials are not sufficiently pore-flexible and do not remodel in situ.

Modifications in the traditional electrospinning process and post-processing conditions have been investigated in the past to develop a polymeric matrix with large pores, improved porosity and pore architecture having the ability to dynamically remodel itself during the regenerative and healing process. One simple and straightforward approach identified in the prior art to increase the pore size of electrospun membranes without compromising the porosity and pore inter-connectivity is increasing the fibers' size [2]. This approach still has the limitation that it does not provide the requisite pore size and dynamism suitable for biological applications pertaining to tissue repair and regeneration. In the prior art, the average pore size in the electrospun membranes developed through traditional electrospinning with nanoscale fibers has been found to be in the range of 500 nm to 2 μm [3], which is too small for cellular transport, and the average pore size in the membranes with microscale fibers has been reported to be in the range of 5-20 μm [4], which is also not adequate for the intended purpose. Controlling the electrospinning parameters [5], fibre distribution using temporary spacers [6] and post-processing techniques such as lyophilisation [7] and salt leaching [6] are the commonly used approaches to increase the pore size and porosity in the developed matrices. In one of the developed technologies, investigators stretched the electrospun membrane product in the transverse and longitudinal directions from 1.5 to 6 times the initial length of the membrane to improve the pore associated parameters [8]. The major limitations associated with this technology was that the membrane was not stretchable beyond 6 times the initial length because of the interfibrillar welds in the membrane. Therefore, adequate pore-size and pore wall flexibility has not been achieved through this technology. This indicate that, till date none of the currently available literature describes an electrospun, fibrous polymeric matrices with sufficient integrity, flexibility, porosity (>80%), having sufficiently large interconnected pores with pore-walls which are openable (flexible). Therefore, this invention addresses the limitation in the currently existing electrospun polymeric matrices by developing highly flexible fibrillar matrices with pore size and geometries suitable for biomedical applications.

### SUMMARY OF THE INVENTION

The present invention discloses an electrospun, flexible, porous, stretchable matrix, comprising mesh of lattice structures of microfibrillar filaments with minimum inter-fibrillar bonding made from polymeric solution comprising one or more synthetic polymers dissolved in one or more organic volatile solvents, characterized in that the synthetic polymers and the organic volatile solvents present in the polymeric solution are in the ratio of 1.8:10 to 6:10 and wherein the matrix has a pore size enlargeable up to 8 times in their size by moving the microfibrillar filaments perpendicular to the longitudinal axis of the microfibrillar filaments.

The present invention also pertains to a method for the preparation of flexible, porous, stretchable matrix, comprising:
i) dissolving one more synthetic polymers in a single organic volatile solvent or combination of organic volatile solvents to obtain a homogeneous fibrous material solution;
ii) dispersing ceramic, metallic or polymeric particles into the solution obtained in step (i) to obtain a composite solution;
iii) loading the homogeneous fibrous material solution obtained in step (i) or the composite solution step (ii) into a syringe or syringes and connecting it to one or more syringe pumps;
iv) electrostatic spinning of the solution through the syringe to produce fibrillar polymeric-jets which is deposited at the fibre bundle collector to obtain parallelly aligned microfiber bundles with minimum inter-fibrillar bonding (0-400/cm²), with 0-60° overlap among the microfibers;
v) creating an air-flow at the inter-phase of fibrillar polymeric-jets and the fibre bundle collector to completely eliminate the solvent from the surface of the parallelly aligned microfiber bundles to avoid inter-fibrillar bonding after collection;
vi) dispersing the microfiber bundles prepared in step (iv) in a direction perpendicular to the longitudinal axis of the fibers, 6-12 times the original width of microfiber bundles using a dispersion unit to obtain a stretch responsive fibrillar matrix.
vii) maintaining the matrix prepared in step (vi) for 30-60 minutes in the fiber dispersion unit and size the matrix for biomedical use.

The membrane and matrix are interchangeably used in the description.

### BRIEF DESCRIPTION OF DRAWINGS

FIG. 1: Schematic diagram of the matrix development process showing: A: Air compressor, B: Air blower with controller, B1 arrows: Air flow from the blower parallel to polymer jet, C: Syringe pump, D: Electrospinning solution loaded syringe, E: High voltage power supply, F: Electrostatically drawn polymer jet, G: high-speed rotating mandrel Fiber bundle collector, G1 arrows: Air flow direction while the collector is running in clock wise direction, H: The solvent evaporation gradient during the fiber bundle collection process, I: Fiber bundle stretching unit loaded with the fiber bundle, J: Processing of the fiber bundle, K: Matrix (Membrane product) loaded cassette.

FIG. 2A: Schematic representation of the stretch depended increase in the size and volume of pores (arrows) observed in the developed matrix (membrane product).

FIG. 2B: Stereomicroscopic images representing the stretch dependent change in pore size, and volume in the developed matrix (membrane product).

FIG. 2C: Ultra-structure of the fibers and the pore in the developed matrix (membrane product).

FIG. 2D: Graphical representation of the diameter of fibers of the matrix (membrane product). The diameter of microfibers were below 50 μm.

FIG. 2E: Quantitative data showing the stretch dependent (up to 1000% strain) porosity increase in the matrix (membrane product).

FIG. 2F: Quantitative data showing the stretch dependent (up to 1000% strain) pore size (in single plane) increase in the matrix (membrane product).

FIG. 2G: Quantitative data showing the stretch dependent (up to 1000% strain) pore size (through the thickness) increase in the matrix (membrane product).

FIG. 2H: Quantitative data showing the stretch dependent (up to 1000% strain) pore-volume increase in the matrix (membrane product).

FIG. 3A: Schematic illustration to show the fibrous matrix (membrane product) porosity and pore-size improving process.

FIG. 3B: The matrix (membrane product) with flexibility and elasticity.

FIG. 4: XPS spectrum indicating the presence of collagen and Extra cellular matrix components in the highly flexible matrix (membrane product).

FIG. 5A: Confocal fluorescence images of human mesenchymal stem cells (hMSCs) attached on the membrane product (matrix) without collagen conjugation at day 1 and day 7 (stained using a Live/dead Cell Viability kit).

FIG. 5B: Confocal fluorescence images of hMSCs growing on the membrane product (matrix) with collagen conjugation at day 1 and 7 (stained using a Live/dead Cell Viability kit).

FIG. 5C: hMSCs viability/metabolic activity on the membrane product (Alamar blue Assay) without collagen conjugation.

FIG. 5D: hMSCs proliferation on the membrane product (DNA content through Hoechst Assay) without collagen conjugation.

FIG. 5E: hMSCs viability/metabolic activity on the membrane product (Alamar blue Assay) with collagen conjugation.

FIG. 5F: hMSCs proliferation on the membrane product (DNA content through Hoechst Assay) with collagen conjugation.

FIG. 6A: Inter-fiber bonding and overlap in the matrix (membrane product) developed with an air flow volume greater than 75 CFM showing low inter-fibre bonding (less than 50/mm²) with 0-60° inter-fiber overlap.

FIG. 6 B: Inter-fiber bonding and overlap in the matrix developed with an air flow volume less than 30 CFM showing more inter-fibre bonding (greater than 500/mm²).

FIG. 7: Morphology and ultra structure of parallelly aligned microfibers without solvent residues in the surface of the fibers, collected at a linear velocity of 5-50 m/s on a high-speed rotating mandrel.

FIG. 8: Images of microfibrous lattice of electrospun matrix (membrane product) unstretched, stretched up to 6 times and 12 times by the stretching unit.

FIG. 9: Images of electrospun matrix (membrane product) unstretched, and membrane product after releasing the 6 times and 12 times stretching by the stretching unit.

FIG. 10: Photographs and scanning electron microscopic images of stretched matrix comprising microfibers made from polylactic acid (PLA), poly lactic-co-glycolic acid (PLGA) and polyurethane (PU) after releasing the stretch.

FIG. 11A: Photographs of matrices made from Polycaprolactone (PCL) dissolved in Chloroform (100%).

FIG. 11B: Photographs of matrices made from Polycaprolactone (PCL) in Chloroform+Trifluroethanol where the concentration of chloroform is less than 70% in the total solvent combination.

FIG. 11C: Photographs of matrices made from Polycaprolactone (PCL) in organic volatile solvent other than Chloroform where it totally lost the elasticity of the membrane and completely deformed.

FIG. 12A: SEM images of the matrix (membrane product) with nTCP (tricalcium phosphate nano-particle) without stretching (0% strain) and after 500% strain.

FIG. 12B: Quantitative data showing the stretch dependent (up to 500% strain) pore size (through the thickness) increase in the matrix (membrane product) with nTCP (tricalcium phosphate nano-particle).

FIG. 12C: Confocal microscopic images of human mesenchymal stem cells (hMSCs) attached on the matrix (membrane product) with nTCP (tricalcium phosphate nano-particle) at day 1 and day 7 (stained using a Live/dead Cell Viability kit).

FIG. 12D: hMSCs viability/metabolic activity on membrane product with nTCP (tricalcium phosphate nano-particle) using Alamar blue Assay.

FIG. 12E: hMSCs proliferation on membrane product with nTCP (tricalcium phosphate nano-particle) using Hoechst DNA quantification Assay.

FIG. 13: fluorescence microscopic images of matrix (membrane product) functionalized with fluorescently tagged antibody.

FIG. 14A: Photograph of microfibrillar sphere-shaped scaffold developed from the matrix (membrane product) as a cell expansion platform.

FIG. 14B: Photograph of microfibrillar disc-shaped scaffold developed from the matrix (membrane product) as a cell expansion platform.

FIG. 14C: Fluorescence microscopic images of human mesenchymal stem cells (hMSCs) attached on the microfibrillar sphere-shaped scaffold developed from the matrix (membrane product) at day 1 (stained using a Live/dead Cell Viability kit).

FIG. 14D: Fluorescence microscopic images of human mesenchymal stem cells (hMSCs) attached on the microfibrillar disc-shaped scaffold developed from the matrix (membrane product) at day 1 (stained using a Live/dead Cell Viability kit).

FIG. 15: Time dependent release of SDF-1α from the matrix soak loaded with SDF-1α.

FIG. 16A: Implantation of the tissue regenerating (periosteum regenerating membrane) at the Critical sized defect (15 mm) in rabbit ulna. Membrane used as sleeves in critical sized bone regeneration.

FIG. 16B: H & E stained sections of rabbit ulna at 10^{th} week.

FIG. 16C: Micro CT Images of defect site at 2^{nd} week and 10 th week showing the bone formation and vasculature.

FIG. 17: Schematic representation of the MSC (Mesenchymal stem cells) isolation/retention and growth facilitating microfibrillar cartridge preliminary prototype.

FIG. 18: MSCs attached and growing in the matrix membrane product from the stromal vascular fraction prepared from adipose tissue.

FIG. 19: Diagram depicting the floatation of matrix membrane product on liquid until degradation.

FIG. 20: Photographs of matrices made from Polycaprolactone (PCL)+polyurethane (PU) in the ratio of 1:1.

FIG. 21: Photographs of matrices made from Polycaprolactone (PCL) containing bovine serum albumin (BSA) particles and gelatin particles.

FIG. 22: Photographs of matrices made from Polycaprolactone (PCL) containing ZnO nanoparticles.

### DETAILED DESCRIPTION

For the purposes of the following detailed description, it is to be understood that the invention may assume various alternative variations and step sequences, except where expressly specified to the contrary. Thus, before describing the present invention in detail, it is to be understood that this invention is not limited to particularly exemplified systems or process parameters that may, of course, vary.

It is also to be understood that the terminology used herein is for the purpose of describing embodiments of the invention only and is not intended to limit the scope of the invention in any manner. The use of examples anywhere in this specification, including examples of any terms discussed herein, is illustrative only and in no way limits the scope and meaning of the invention or any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions, will control. It must be noted that, as used in this specification and the appended claims, the singular forms “a,” “an” and “the” include plural referents unless the content clearly dictates otherwise. The terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

As used herein, the terms “comprising” “including” “having” “containing” “involving” and the like are to be understood to be open-ended, i.e., to mean including but not limited to.

The present invention discloses a matrix, comprising mesh of lattice structures of microfibrillar filaments made from polymeric solution comprising one or more synthetic polymer dissolved in one or more organic volatile solvent, characterized in that the synthetic polymer and the organic volatile solvent present in the polymeric solution are in the ratio of 1.8:10 to 6:10 and wherein the matrix has a pore size enlargeable up to 8 times in their size by moving the microfibrillar filaments perpendicular to the longitudinal axis of the microfibrillar filaments.

In various embodiments of the matrix, the synthetic polymer is selected from one of polycaprolactone, polylactic acid, poly lactic-co-glycolic acid or polyurethane or a combination thereof.

In various embodiments of the matrix, the combination of polymers comprises at least two synthetic polymers selected from Polycaprolactone, Polylactic acid, Poly lactic-co-glycolic acid and polyurethane, in the ratio up to 1:1.

In one of the preferable embodiment of the matrix, the synthetic polymer is polycaprolactone.

In various embodiments of the matrix, the one or more organic volatile solvents is one of chloroform, or combination of chloroform and methanol or combination of chloroform and fluorinated alcohol selected from Trifluoroethanol or Hexafluoroisopropanol wherein the amount of chloroform is a minimum of 70% in the final solvent mixture.

In various embodiments of the matrix, ceramic, metallic or polymeric particles or their combinations are embedded fully or partly within, on the surface or on both of the micro-fibrillar filaments.

In various embodiments of the matrix, the ceramic particles comprises of Tri-calcium phosphate, or Hydroxyapatite.

In various embodiments of the matrix, the metallic particles comprises of Zinc oxide, Iron oxide.

In various embodiments of the matrix, the polymeric particles comprises of Gelatin, Albumin, Chitosan.

In various embodiments of the matrix, the ceramic, metallic or polymeric particles have a particle size ranging from 100 nm to 2 micrometers.

In an aspect of the invention, the size of the pores present in the matrix on stretching is in the range of 10-1250 μm in a single plane and 10-250 μm through-the-thickness of the membrane.

In various embodiments of the matrix, the surface of the micro-fibrillar filaments is functionalized with biomolecules selected from extracellular matrix (ECM) components including collagen, fibronectin, decellularized ECM, antigen specific proteins such as antibodies including CD73, CD90 and CD105 thereof.

In various embodiments of the matrix, the surface of the micro-fibrillar filaments is functionalized with biomolecules by covalent cross-linking method to increase the functionality of matrix in facilitating adhesion and regeneration.

In various embodiments of the matrix, the diameter of micro-fibrillar filaments ranges from 1-50 μm.

In various embodiments of the matrix, it has an elasticity and flexibility of up to 500% strain.

In various embodiments of the matrix, it is loaded with bioactive factors such as SDF-1 α (Stromal cell-derived factor 1 alpha) for imparting bioactivity.

In various embodiments, the matrix is employed as one of sleeves and pockets at the defective site for tissue regeneration, 3D cell culture platform, cell sorting and/or expansion apparatus to minimize the complex in vitro procedures during stem cells isolation and expansion to improve the success of transplantation.

In yet another embodiment, the matrix is a scaffold to support cell migration and vasculature generation across the matrix.

In further embodiment, the matrix is an encapsulating material to support floating of one or more components, in the form of membranes or hydrogels loaded with drugs or bioactive factors for delivering into a liquid media or localised site.

Another aspect of the invention pertains to a method for the preparation of matrix, comprising:
i) dissolving one or more synthetic polymers in a single organic volatile solvent or combination of organic volatile solvents to obtain a homogeneous fibrous material solution;
ii) dispersing ceramic, metallic or polymeric particles into the solution obtained in step (i) to obtain a composite solution;
iii) loading the homogeneous fibrous material solution obtained in step (i) or the composite solution step (ii) into a syringe or syringes and connecting it to one or more syringe pumps;
iv) electrostatic spinning of the solution through the syringes to produce fibrillar polymeric-jets which is deposited at the fiber bundle collector to obtain parallelly aligned microfiber bundles with minimum inter-fibrillar bonding (0-400/cm²), with 0-60° overlap among the microfibers;
v) creating an air-flow at the inter-phase of fibrillar polymeric-jets and the fiber bundle collector to completely eliminate the solvent from the surface of the parallelly aligned microfiber bundles;
vi) dispersing the microfiber bundles prepared in step (iv) in a direction perpendicular to the longitudinal axis of the fibers, 6-12 times the original width of microfiber bundles using a dispersion unit to obtain a stretch responsive fibrillar matrix.
vii) maintaining the matrix prepared in step (vi) for 30-60 minutes in the fiber dispersion unit and size the membrane for biomedical use.

In an embodiment of the method for the preparation of matrix, the electrostatic spinning in step (iv) is done at an applied voltage of at least 7.5 kV.

In an embodiment of the method for the preparation of matrix, the rate of the flow of homogeneous fibrous material solution or composite solution is at least 2.5 mL/hr.

In an embodiment of the method for the preparation of matrix, the air flow volume near the mandrel (G) of the electrospinning apparatus is 60 CFM.

In various embodiments of the method for the preparation of matrix, the viscosity of homogeneous fibrous material solution or composite solution is in the range of 1500 to 6000 Cp.

In various embodiments of the method for the preparation of matrix, the microfiber bundles without solvent residues on their surface are collected in a parallelly aligned fashion at a linear velocity of 5-50 m/second on a high-speed rotating mandrel;

In an aspect of the invention, the electrostatic spinning is carried out in electrospinning apparatus comprising Air compressor (A); Air blower with controller (B); Syringe pump (C); Electrospinning solution loaded syringe (D) with the needle; High voltage power supply (E); Electrostatically drawn polymer jet (F); Fiber bundle collector, which is a high-speed rotating mandrel (G); Fiber bundle stretching unit/Fiber dispersion unit loaded with the fiber bundle (I).

In an embodiment of the electrospinning apparatus, the distance between the needle (D) to the mandrel (G) of electrospinning apparatus is at least 5 cm.

In another aspect the porosity of the membrane increases from 50% to 98%.

The matrix of the invention is highly stretchable and has large pore sizes with elastic pore walls and pore sizes that is tuned by transverse mechanical “dispersion” of the fibers without losing membrane integrity.

In a particular embodiment of the method of preparing the matrix, a strain of up to 1000% is introduced with custom-developed Fiber bundle stretching unit/Fiber dispersion unit on the microfibers to attain the required dispersion, porosity and pore size in the membrane.

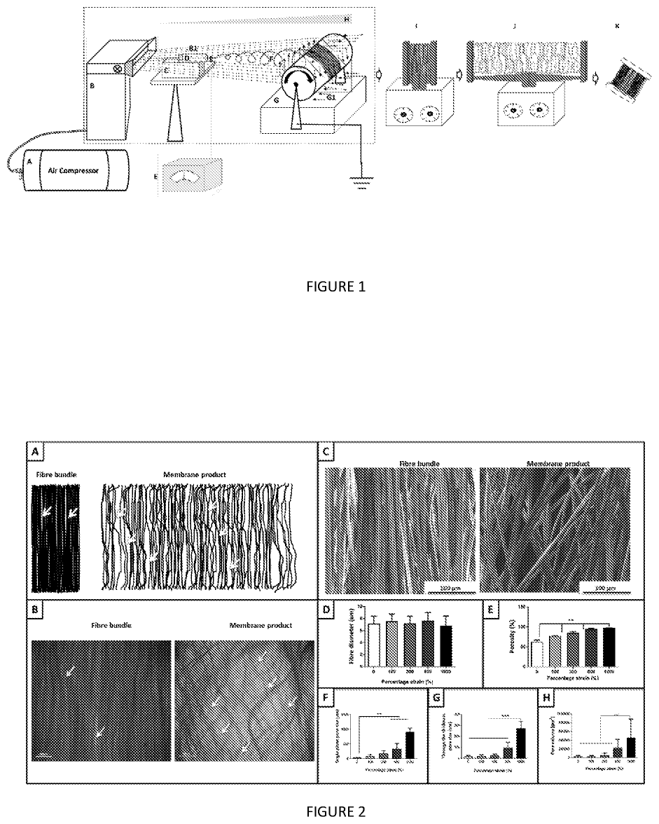

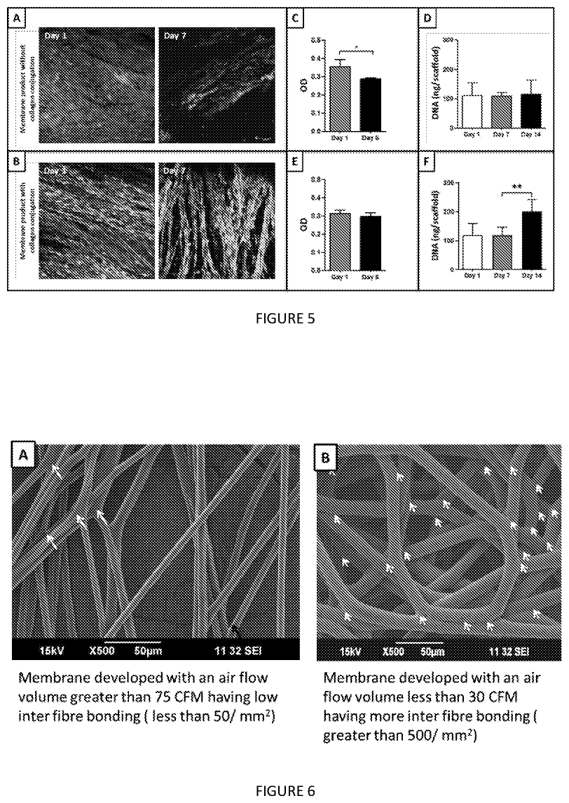

In a particular embodiment of the method of preparing the matrix, controlled flow of air from the Air blower with controller (B) is in the direction of the polymeric jet which facilitates removal of the organic solvent from the developed membrane/matrix resulting in parallelly aligned fibrillar structural architecture with minimum inter fibrillar bonding as indicated by arrows (G1) in FIG. 1. Thus the electrostatic spinning process weakens the fiber-fiber interconnects (or welds) during electrospinning deposition of polymeric jets by controlling the directionality of air-flow and substrate rotation-controlled deposition system that prevents strong interconnects formation (FIG. 6).

In an aspect dispersion (spreading the fibers in the bundle) of the matrix is brought about by stretching in a direction perpendicular to the longitudinal axis of the fibers so as to disperse the fibers without any tearing or failure of the membrane using Fiber bundle stretching unit/Fiber dispersion unit. The dispersion process opens up the pores, increases the pore volume and size eliminates some of the interconnects and provides the requisite architecture. This critical “dispersion” step creates a highly stretchable membrane with the stretch of up to 12 times in width giving not only large pores but also a structure that can give openable pore walls that allow for porosity remodelling during use. The dispersion process is controlled so as not to fracture the matrix but only improve porosity and pore size.

The resulting matrix having a pore size enlargeable up to 10-12 times preferably 8 times in their size while at the same time retaining its structural integrity, could facilitate, by structural remodeling, the permeation of cells and capillaries across the membrane during tissue regeneration or tissue growth.

In another aspect, the electrospun microfibrous porous stretchable matrix is used in, but are not limited to periosteum regeneration in segmental defects, wound dressing, 3D cell culture substrates, micro-carriers for cell expansion systems, and cell-isolation devices.

In another aspect the electrospun microfibrous porous stretchable matrix is used in cell isolation, sorting and/or expansion apparatus to minimize the complex in vitro procedures during stem cells isolation and expansion to improve the success of transplantation.

### EXAMPLES

Without limiting the scope of the present invention as described above in any way, the present invention has been further explained through the examples provided below. Materials used in the invention such as polymers were obtained from Puralite, USA, Envisiontec, Germany and Carbion Netherlands. Other agents were obtained from MERCK, INDIA, SIGMA, USA, THERMOFISHER SCIENTIFIC, USA, HIMEDIA, INDIA

### Example 1: Preparation of Electrospun Microfibrous Porous Stretchable Membrane/Matrix

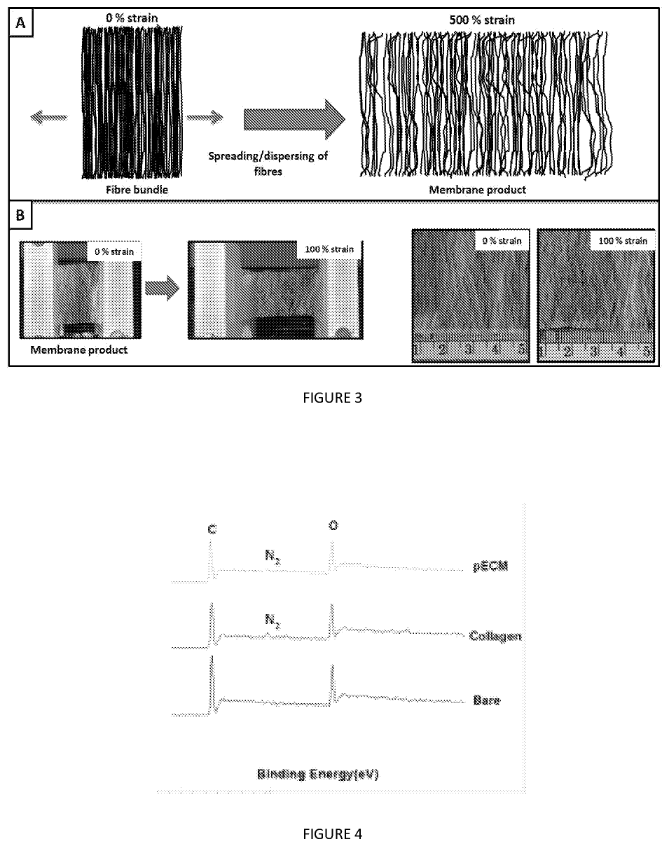

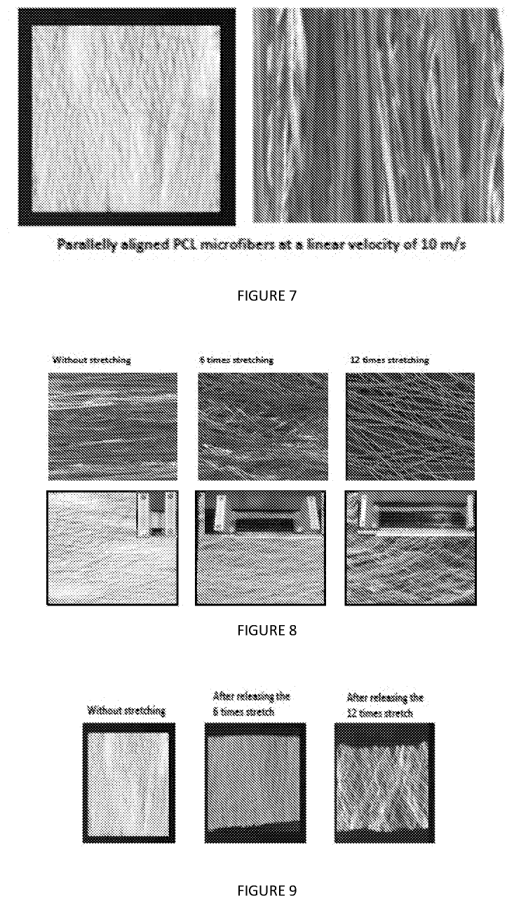

For preparing the homogeneous fibrous material solution for electrostatic spinning, 5.8 gm of Polycaprolactone PCL (MW: 120 kDa) was dissolved in 10 mL of chloroform. The homogeneous fibrous material solution was electrospun from a 20 ml syringe (attached with a blunt end needle) at a flow rate of 4 ml/hr within a chemical hood. An applied voltage of 20 kV was maintained at the needle tip using a high voltage power supply (Gamma high voltage). The microfibers were collected at a linear velocity of 10 m/s on a rotating mandrel for 20 minutes by keeping the tip to mandrel distance at 20 cm under an airflow volume up to 190 CFM (cubic feet per minute). The schematic of the electrospinning setup is shown in FIG. 1 and the increase in pore size were represented as a schematic diagram in FIG. 2A. SEM image (FIG. 2C) shows the micro fibrillar architecture of the matrix. The developed membrane is shown in FIG. 7.

### Example 2: Porosity Determination of the Matrix

Porosity of the matrix in response to different strain (up to 1000%) was analysed (FIG. 2E). The porosity of the micro fibrous PCL membrane was measured using gravimetric method. The thickness of membranes was measured using Vernier calipers. Density of the membrane (ρ_{membrane}) was calculated based on its mass and volume. Density of the material (ρₘₐₜₑᵣᵢₐₗ) was 1.145 g/cc., as mentioned in the product catalogue. The porosity was then calculated according to the equation,

$$∈ = 1 - \frac{{ρ}_{membrane}}{{ρ}_{material}}$$

The same procedure with variations in parameters was done with other polymer types and combinations.

### Example 3: Preparation of PLA Electrospun Microfibrous Porous Stretchable Membrane/Matrix

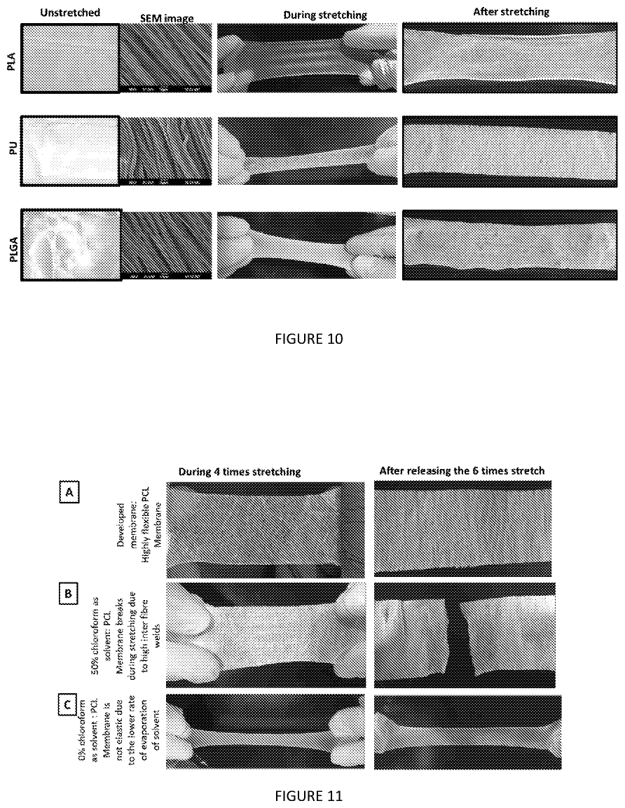

For preparing the homogeneous PLA fibrous material solution for electrostatic spinning, 4.8 gm of Poly lactic acid PLA (MW: 50 kDa) was dissolved in 10 mL of chloroform. The homogeneous fibrous material solution was electrospun from a 20 ml syringe (attached with a blunt end needle) at a flow rate of 4 ml/hr within a chemical hood. An applied voltage of 12 kV was maintained at the needle tip using a high voltage power supply (Gamma high voltage). The microfibers were collected at a linear velocity of 10 m/s on a rotating mandrel for 30 minutes by keeping the tip to mandrel distance at 15 cm under an airflow volume up to 190 CFM (cubic feet per minute). The developed PLA membrane and its flexibility was shown in FIG. 10.

### Example 4: Preparation of PU Electrospun Microfibrous Porous Stretchable Membrane/Matrix

For preparing the homogeneous PU fibrous material solution for electrostatic spinning, 1.5 gm of carbothene (PU) (MW: 88 kDa) was dissolved in 10 mL of chloroform. The homogeneous fibrous material solution was electrospun from a 20 ml syringe (attached with a blunt end needle) at a flow rate of 5 ml/hr within a chemical hood. An applied voltage of 12 kV was maintained at the needle tip using a high voltage power supply (Gamma high voltage). The microfibers were collected at a linear velocity of 10 m/s on a rotating mandrel for 30 minutes by keeping the tip to mandrel distance at 20 cm under an airflow volume up to 190 CFM (cubic feet per minute). The developed PU membrane and its flexibility was shown in FIG. 10.

### Example 5: Preparation of PLGA Electrospun Microfibrous Porous Stretchable Membrane/Matrix

For preparing the homogeneous PLGA fibrous material solution for electrostatic spinning, 5.5 gm of poly lactic glycolic acid (PLGA) (75:25) was dissolved in 10 mL of chloroform. The homogeneous fibrous material solution was electrospun from a 20 ml syringe (attached with a blunt end needle) at a flow rate of 4 ml/hr within a chemical hood. An applied voltage of 12 kV was maintained at the needle tip using a high voltage power supply (Gamma high voltage). The microfibers were collected at a linear velocity of 18 m/s on a rotating mandrel for 30 minutes by keeping the tip to mandrel distance at 20 cm under an airflow volume up to 190 CFM (cubic feet per minute). The developed PLGA membrane and its flexibility was shown in FIG. 10.

### Example 6: Preparation of Electrospun Microfibrous Porous Stretchable Membrane/Matrix Using Combination of Volatile Organic Solvents

For preparing the homogeneous fibrous material solution for electrostatic spinning containing two organic solvents, 5.8 gm of Polycaprolactone PCL (MW: 120 kDa) was dissolved in a mixture of chloroform and TFE (Trifluoroethanol) in the ratio of 1:1 (5 mL chloroform and 5 mL TFE). The homogeneous fibrous material solution was electrospun from a 20 mL syringe (attached with a blunt end needle) at a flow rate of 4 ml/hr within a chemical hood. An applied voltage of 20 kV was maintained at the needle tip using a high voltage power supply (Gamma high voltage). The microfibers were collected at a linear velocity of 10 m/s on a rotating mandrel for 30 minutes by keeping the tip to mandrel distance at 20 cm under an airflow volume up to 190 CFM (cubic feet per minute). The developed membrane breaks when we apply 500% strain (FIG. 11B).

### Example 7: Preparation of Electrospun Microfibrous Porous Stretchable Membrane/Matrix without Chloroform

For preparing the homogeneous fibrous material solution for electrostatic spinning containing 0% chloroform, 5.8 gm of Polycaprolactone PCL (MW: 120 kDa) was dissolved in 10 mL TFE (Trifluoroethanol). The homogeneous fibrous material solution was electrospun from a 20 ml syringe (attached with a blunt end needle) at a flow rate of 4 ml/hr within a chemical hood. An applied voltage of 20 kV was maintained at the needle tip using a high voltage power supply (Gamma high voltage). The microfibers were collected at a linear velocity of 10 m/s on a rotating mandrel for 30 minutes by keeping the tip to mandrel distance at 20 cm under an airflow volume up to 190 CFM (cubic feet per minute). The developed membrane is not elastic/flexible due to the lower rate of solvent evaporation (FIG. 11C).

### Example 8: Preparation of Electrospun Microfibrous Porous Stretchable Membrane/Matrix Containing Tricalcium Phosphate Nanoparticles

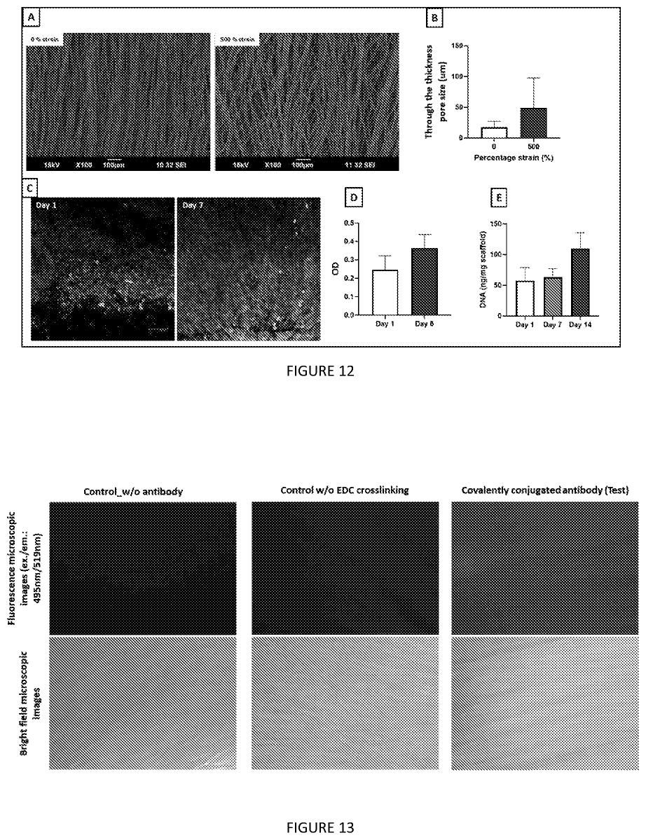

For preparing the fibrous material solution containing tricalcium phosphate nanoparticles (nTCP) for electrostatic spinning, as a first step 2.5 gm of nTCP were sonicated in 5 mL of chloroform and 5.8 gm of Polycaprolactone PCL (MW: 120 kDa) was dissolved in 5 mL of chloroform. The homogeneous fibrous material solution was prepared by mixing the 5 mL nTCP and PCL solution together and electrospun from a 20 mL syringe (attached with a blunt end needle) at a flow rate of 4 ml/hr within a chemical hood. An applied voltage of 20 kV was maintained at the needle tip using a high voltage power supply (Gamma high voltage). The microfibers were collected at a linear velocity of 10 m/s on a rotating mandrel for 45 minutes by keeping the tip to mandrel distance at 10 cm under an airflow volume up to 190 CFM (cubic feet per minute). The stretchability of developed membrane is shown in FIG. 12.

### Example 9: Preparation of Electrospun Microfibrous Porous Stretchable Membrane/Matrix Containing Two Different Synthetic Polymers

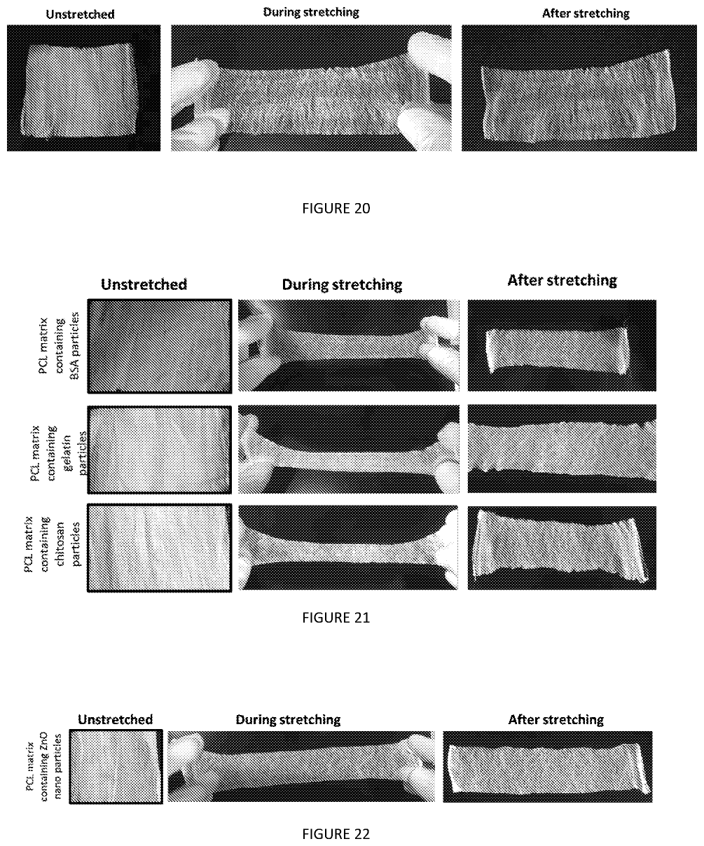

For preparing the fibrous material solution containing two different synthetic polymer for electrostatic spinning, as a first step 750 mg of carbothene (PU) (MW: 88 kDa) was dissolved in 5 mL of chloroform. 2.9 gm of Polycaprolactone PCL (MW: 120 kDa) was dissolved in 5 mL of chloroform. The homogeneous fibrous material solution was prepared by mixing the 5 mL PU and PCL solution together and electrospun from a 20 mL syringe (attached with a blunt end needle) at a flow rate of 4 ml/hr within a chemical hood. An applied voltage of 12 kV was maintained at the needle tip using a high voltage power supply (Gamma high voltage). The microfibers were collected at a linear velocity of 10 m/s on a rotating mandrel for 30 minutes by keeping the tip to mandrel distance at 10 cm under an airflow volume up to 190 CFM (cubic feet per minute). The stretchability of developed membrane is shown in FIG. 20.

### Example 10: Preparation of Electrospun Microfibrous Porous Stretchable Membrane/Matrix Containing Polymeric Particles

For preparing the fibrous material solution containing polymeric particles (gelatin/bovine serum albumin (BSA)/chitosan particles) for electrostatic spinning, as a first step 200 mg of polymeric particle was mixed in 10 mL of chloroform for 15 minutes. 5.8 gm of Polycaprolactone PCL (MW: 120 kDa) was dissolved in 10 mL of chloroform containing polymeric particle. The homogeneous fibrous material solution was electrospun from a 20 mL syringe (attached with a blunt end needle) at a flow rate of 4 ml/hr within a chemical hood. An applied voltage of 12 kV was maintained at the needle tip using a high voltage power supply (Gamma high voltage). The microfibers were collected at a linear velocity of 10 m/s on a rotating mandrel for 30 minutes by keeping the tip to mandrel distance at 10 cm under an airflow volume up to 190 CFM (cubic feet per minute). The stretchability of developed membrane is shown in FIG. 21.

### Example 11: Preparation of Electrospun Microfibrous Porous Stretchable Membrane/Matrix Containing ZnO Nanoparticles

For preparing the fibrous material solution containing ZnO nanoparticle for electrostatic spinning, as a first step 200 mg of ZnO nanoparticle was sonicated in 10 mL of chloroform for 30 minutes. 5.8 gm of Polycaprolactone PCL (MW: 120 kDa) was dissolved in 10 mL of chloroform containing ZnO nanoparticle. The homogeneous fibrous material solution was electrospun from a 20 mL syringe (attached with a blunt end needle) at a flow rate of 4 ml/hr within a chemical hood. An applied voltage of 12 kV was maintained at the needle tip using a high voltage power supply (Gamma high voltage). The microfibers were collected at a linear velocity of 10 m/s on a rotating mandrel for 30 minutes by keeping the tip to mandrel distance at 10 cm under an airflow volume up to 190 CFM (cubic feet per minute). The stretchability of developed membrane is shown in FIG. 22.

### Example 12: Preparation of Functionalized Electrospun Microfibrous Porous Stretchable Membranes

The developed membranes pre-wetted using alcohol gradient are kept in IN NaOH solution for 3 h to alkali hydrolysis. After hydrolysis, the membranes are neutralized using IN HCL followed by repeated washing in distilled water. Then the membranes were immersed in conjugation buffer solution containing EDC (1-Ethyl-3-(3-dimethyl aminopropyl) carbodiimide) and NHS (N-Hydroxysuccinimide) in the ratio of 2:1 for 1 h at room temperature and washed the membranes using fresh conjugation buffer without EDC and NHS to remove the residual reagents. Then the following steps will be followed for each biomolecule functionalization:
(i) For collagen functionalization, the membranes were treated with collagen solution with a concentration of 50 μg/ml for 24 h at 4° C. After conjugation, the membranes were dried and then washed in deionised water thoroughly to ensure complete removal of free collagen. Washed membranes were then air dried and stored.
(ii) For fibronectin functionalization, the membranes were treated with fibronectin solution with a concentration of 50 μg/ml for 24 h at 4° C. After conjugation, the membranes were dried and then washed in deionised water thoroughly to ensure complete removal of free fibronectin. Washed membranes were then air dried and stored.
(iii) For decellularised ECM functionalization, the membranes were treated with decellularised ECM solution with a concentration of 50 μg/ml for 24 h at 4° C. After conjugation, the membranes were dried and then washed in deionised water thoroughly to ensure complete removal of free decellularised ECM. Washed membranes were then air dried and stored.
(iv) For antibodies (CD73, CD90 and CD105) functionalization, the membranes were treated with antibodies solution with a maximum of 1:50 dilution for 24 h at 4° C. After conjugation, the membranes were dried and then washed in deionised water thoroughly to ensure complete removal of free antibodies. Washed membranes were then air dried and stored.

The presence of bioactive molecule such as collagen and decellularised ECM on the surface of the membrane was confirmed using XPS analysis. The XPS spectrum showed a nitrogen peak along with carbon and oxygen, confirming the presence of collagen and decellularised ECM on the surface of the membrane (FIG. 4) Also, the fluorescence microscopic images (FIG. 13) confirms the presences of fluorescently conjugated antibody in the matrix (membrane product).

### Example 13: In Vitro Experiments on the Use of the Membranes

Human adipose derived mesenchymal stem cells (hMSCs) were seeded on to the membrane at two different densities such as 2×10⁴ cells/mm² and 1×10×4 cells/mm² to evaluate their viability and proliferation on the matrix. The viability of the cells were evaluated at day 1 and 7 and proliferation was evaluated up to 14 days. The developed matrix with collagen conjugation showed good biocompatibility, and human mesenchymal stem cells (hMSCs) attached and aligned in the direction of fibre orientation and proliferated over time (FIG. 5A-B). Cytotoxicity/metabolic activity of the cells growing in the collagen conjugated membrane product showed no significant difference between days 1 and 6 (FIGS. 5C and 5E), and hMSCs showed proliferation within 14 days of culture (FIGS. 5D and 5F).

### Example 14: Development of Cell Culture/Expansion Platforms Using the Matrix

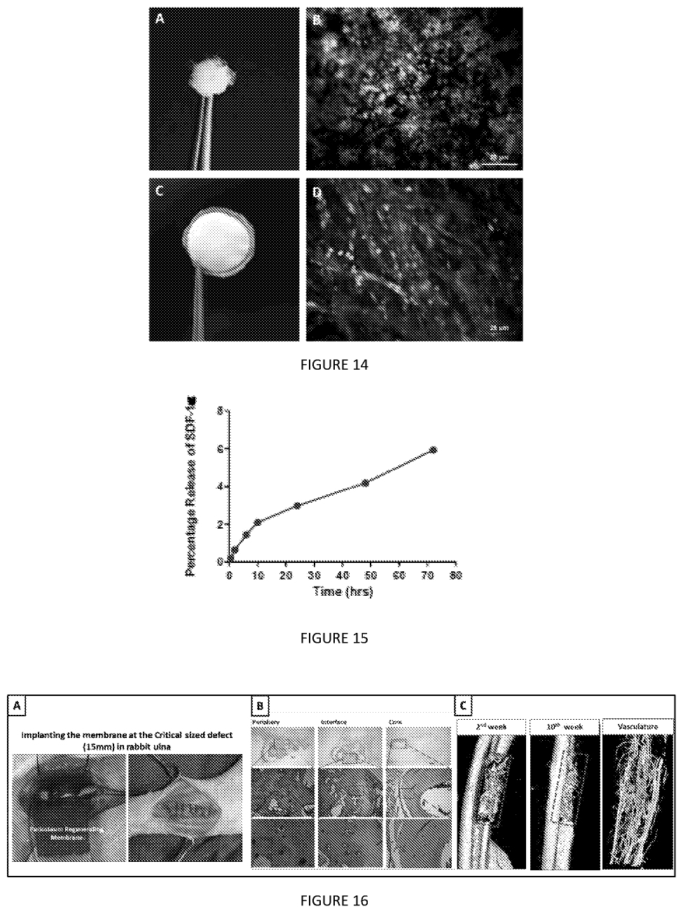

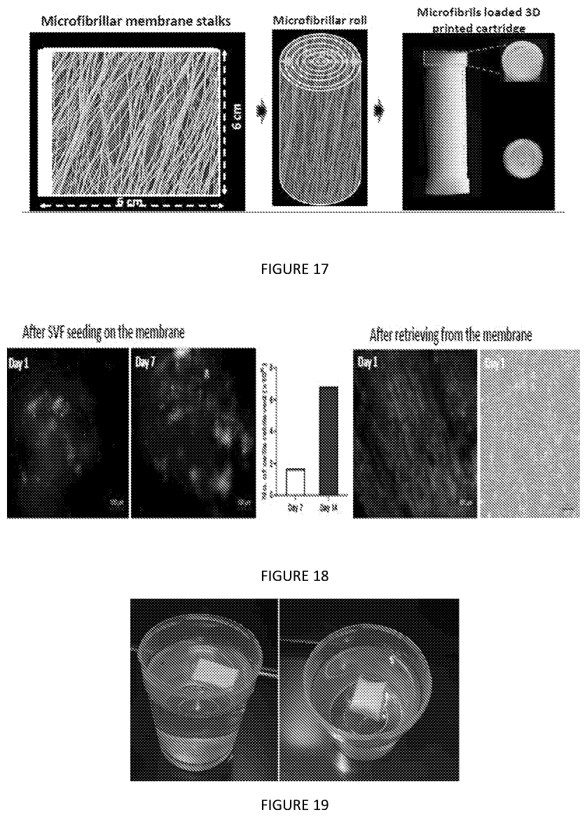

(i) Microfibrillar sphere shaped scaffold
   The PCL polymeric solution were electrospun for 3 hrs and the developed membranes were then stretched from both ends perpendicular to the fibre alignment until the membrane width became 7 times its initial width. The membrane was maintained under the stretched condition for 15 minutes. Then, the stretched membranes were placed inside a custom-made scaffold fabrication mold and a load of 1 ton was applied for 2 minutes using a hydraulic press, to obtain a string of spheres. Individual spheres shaped scaffolds were separated from the string of strips using a surgical blade (FIG. 14A).
(ii) Microfibrillar disc shaped scaffold:
   The PCL polymeric solution were electrospun for 2 hrs and the developed membranes were then stretched from both ends perpendicular to the fibre alignment until the membrane width became 10 times its initial width. Four layers of the stretched membranes were stacked one above the other to get a thickness of ˜1 mm for the Microfibrillar sphere shaped scaffold. Using a custom-made mold, a load of 2 tons was applied on the developed scaffold's peripheral boundary to get a 1 mm wide fused area leaving a microfibrillar-matrix area of ˜28 mm² at the centre. Further, individual scaffolds were obtained using a custom-made 8 mm biopsy punch (FIG. 14C).

Human adipose derived mesenchymal stem cells (hMSCs) were seeded on to the scaffolds with a seeding density of 1×10⁶ cells/scaffold to evaluate their viability on the scaffold. The viability of the cells were evaluated at day 1. Both the developed scaffolds showed good biocompatibility, and majority of the human mesenchymal stem cells (hMSCs) were viable (FIGS. 14B and 14D).

### Example 15: SDF-1α Loading on the Matrix

The developed membranes pre-wetted using alcohol gradient are kept in with SDF-1α solution with a concentration of 100 ng/ml for 24 h at 4° C. After conjugation, the membranes were dried and then washed in deionised water thoroughly to ensure complete removal of free SDF-1α. Washed membranes were then air dried and stored. The SDF-1α coated matrix shows an initial burst release and then sustained release of SDF-1α (FIG. 15).

### Example 16: Use of Developed Matrix as a Sleeve at Critical Sized Bone Defect

A 15 mm defect was made in rabbit ulna and the developed matrix were used as a sleeve to hold autologous bone chips in it. The membrane were wrapped around the bone defect and filled with crushed autologous bone (FIG. 16A). Then suture the membrane to hold the bone chips inside properly. After 10 weeks, the animals were euthanized and collected the defect bone and performed histology (FIG. 16B). New bone formation was observed at the defect interface region. The bone formation was confirmed using microCT imaging (FIG. 16C). Comparison of 2nd week, 10th week microCT images shows bone formation. Also, vasculature at the defect site was analysed using microCT imaging.

### Example 17: Development of Cell Isolation/Retention and Growth Facilitating Microfibrillar Cartridge Preliminary Prototype

Rolls of the membrane product prepared by stalking 10 layers of the matrix was manually loaded within a 3D printed polymeric cartridge with a volume of 400 cm³. The membrane product loaded cartridge prototype (microfibrillar cartridge) was sterilized using ethylene oxide and were used for the cell isolation and expansion.

### Example 18: Isolation/Retention and Growth of hMSCs (Human Mesenchymal Stem Cells) Using Microfibrillar Cartridge

Stromal vascular fraction (SVF) was obtained from the human lipoaspirate by collagenase treatment. The microfibrillar cartridge was prewetted by incubating in phosphate buffered saline and SVF (2 mL/cartridge) was loaded on top of the prewetted microfibrillar cartridge. The cell loaded microfibrillar cartridge was incubated for 30-45 minutes at 37° C. and then the adhered cells were cultured for 14 days. The viability of the cells grown on the matrix were evaluated at day 1 and day 7 (FIG. 18).

### Example 19: Developing Floating Devices that can Deliver Drugs or Bioactive Factors

Hydrogels made out of alginate, and polymeric membranes made out of gelatin and PCL were loaded with drugs and were made as floating devices by encapsulating with the developed membrane product. Encapsulation was achieved by heat-sealing the edges of the membrane product. The membrane product was capable of accommodating 50 times the weight of the membrane within the device and remained floated for a period of 8 weeks (FIG. 19).

### Advantages

1. Matrix comprising mesh of lattice structures of microfibrillar filaments with 0-60° interfiber overlap and low interfiber bonding 0-400/cm² having pores of up to 1250 μm in size in a given plane and pore channel sizes through-thickness of up to 250 μm.
2. Matrix is highly stretchable from 6-12 times in width, preferably 8 times in width.
3. Matrix is highly flexible.
4. Matrix with sufficient integrity for use in bone regeneration applications, implant sleeves, 3D cell culture, cell sorting and haemostatic dressings.
5. Matrix bio-functionalized by immobilization of biomolecules on the surface of the fibers before or after post processing facilitate biofunctions such as cell attachment, wound healing, clot formation, cell expansion and so on.
6. Matrix is suturable and cytocompatible.
7. Matrix exhibits good mechanical integrity.

### REFERENCES

[1] A. Guimaires, A. Martins, E. D. Pinho, S. Faria, R. L. Reis, and N. M. Neves, “Solving cell infiltration limitations of electrospun nanofiber meshes for tissue engineering applications,” Nanomedicine, vol. 5, no. 4, pp. 539-554, 2010, doi: 10.2217/nnm.10.31.
[2] A. Balguid, A. Mol, M. H. Van Marion, R. A. Bank, C. V. C. Bouten, and F. P. T. Baaijens, “Tailoring fiber diameter in electrospun poly(ε-Caprolactone) scaffolds for optimal cellular infiltration in cardiovascular tissue engineering,” Tissue Eng.—Part A, vol. 15, no. 2, pp. 437-444, 2009, doi: 10.1089/ten.tea.2007.0294.
[3] H. Li, C. Huang, X. Jin, and Q. Ke, “An electrospun poly(ε-caprolactone) nanocomposite fibrous mat with a high content of hydroxyapatite to promote cell infiltration,” RSC Adv., vol. 8, no. 44, pp. 25228-25235, 2018, doi: 10.1039/c8ra02059k.
[4] P. R. Cortez Tornello, P. C. Caracciolo, J. I. Igartúa Roselló, and G. A. Abraham, “Electrospun scaffolds with enlarged pore size: Porosimetry analysis,” Mater. Lett., vol. 227, pp. 191-193, 2018, doi: 10.1016/j.matlet.2018.05.072.
[5] K. Sisson, C. Zhang, M. C. Farach-Carson, D. B. Chase, and J. F. Rabolt, “Fiber diameters control osteoblastic cell migration and differentiation in electrospun gelatin,” J. Biomed. Mater. Res.—Part A, vol. 94, no. 4, pp. 1312-1320, 2010, doi: 10.1002/jbm.a.32756.
[6] J. Wu and Y. Hong, “Enhancing cell infiltration of electrospun fibrous scaffolds in tissue regeneration,” Bioactive Materials, vol. 1, no. 1. pp. 56-64, 2016, doi: 10.1016/j.bioactmat.2016.07.001.
[7] M. Simonet, O. D. Schneider, P. Neuenschwander, and W. J. Stark, “Ultraporous 3D polymer meshes by low-temperature electrospinning: Use of ice crystals as a removable void template,” Polym. Eng. Sci., vol. 47, no. 12, pp. 2020-2026, 2007, doi: 10.1002/pen.20914.
[8] KR101853283B1/JP6140295B2/WO2014075388A1/EP2921136B1/BR112015009502B1/IN2015DN01362A

1. A matrix, comprising mesh of lattice structures of microfibrillar filaments made from polymeric solution comprising one or more synthetic polymer dissolved in one or more organic volatile solvent, characterized in that the synthetic polymer and the organic volatile solvent present in the polymeric solution are in the ratio of 1.8:10 to 6:10 and wherein the matrix has a pore size enlargeable up to 8 times in their size by moving the microfibrillar filaments perpendicular to the longitudinal axis of the microfibrillar filaments.

2. The matrix as claimed in claim 1, wherein the synthetic polymer is selected from one of polycaprolactone, polylactic acid, poly lactic-co-glycolic acid or polyurethane or a combination thereof.

3. The matrix as claimed in claim 2, wherein combination of polymers comprises at least two synthetic polymers selected from Polycaprolactone, Polylactic acid, Poly lactic-co-glycolic acid and polyurethane, in the ratio up to 1:1

4. The matrix as claimed in claim 1, wherein the one or more organic volatile solvents is one of chloroform, or combination of chloroform and methanol or combination of chloroform and fluorinated alcohol selected from Trifluoroethanol or Hexafluoroisopropanol wherein the amount of chloroform is a minimum of 70% in the final solvent mixture.

5. The matrix as claimed in claim 1, wherein the size of the pores is in the range of 10-1250 μm in a single plane and 10-250 μm through-the-thickness of the membrane.

6. The matrix as claimed in claim 1, wherein the ceramic, metallic or polymeric particles or their combinations are embedded within or on the surface or both of the microfibrillar filaments.

7. The matrix as claimed in claim 6, wherein the ceramic particles comprises of Tri-calcium phosphate, or Hydroxyapatite.

8. The matrix as claimed in claim 6, wherein the metallic particles comprises of Zinc oxide, Iron oxide.

9. The matrix as claimed in claim 6, wherein the polymeric particles comprises of Gelatin, Albumin, Chitosan.

10. The matrix as claimed in claim 6, wherein the ceramic, metallic or polymeric particles has a particle size ranging from 100 nm to 2 micrometers.

11. The matrix as claimed in claim 1, wherein the surface of the microfibrillar filaments is functionalized with biomolecules selected from extracellular matrix components including collagen, fibronectin, decellularized ECM, antigen specific proteins such as antibodies including CD73, CD90 and CD105 thereof.

12. The matrix as claimed in claim 1, wherein the diameter of microfibrillar filaments ranges from 1-50 μm.

13. The matrix as claimed in claim 1, wherein it has an elasticity and flexibility of up to 500% strain.

14. The matrix as claimed in claim 1, wherein the matrix is loaded with bioactive factors such as SDF-1 α for imparting bioactivity.

15. The matrix as claimed in claim 1, wherein the matrix is one of sleeves and pockets at the defective site for tissue regeneration, 3D cell culture platform, cell sorting and/or expansion apparatus to minimize the complex in vitro procedures during stem cells isolation and expansion to improve the success of transplantation.

16. The matrix as claimed in claim 1, wherein the matrix is a scaffold to support cell migration and vasculature generation across the matrix.

17. The matrix as claimed in claim 1, wherein the matrix is an encapsulating material to support floating of one or more components, in the form of membranes or hydrogels loaded with drugs or bioactive factors for delivering into a liquid media or localised site.

18. A method for the preparation of matrix, comprising:
i) dissolving one more synthetic polymer in a single organic volatile solvent or combination of organic volatile solvents to obtain a homogeneous fibrous material solution;
ii) dispersing ceramic, metallic or polymeric particles into the solution obtained in step (i) to obtain a composite solution;
iii) loading the homogeneous fibrous material solution obtained in step (i) or the composite solution step (ii) into a syringe or syringes and connecting it to one or more syringe pumps;
iv) electrostatic spinning of the solution through the syringe pumps to produce fibrillar polymeric-jets which is deposited at the fiber bundle collector to obtain parallelly aligned microfiber bundles with minimum inter-fibrillar bonding (0-400/cm²), with 0-60° overlap among the microfibers;
v) creating an air-flow at the inter-phase of fibrillar polymeric-jets and the fiber bundle collector to completely eliminate the solvent from the surface of the parallelly aligned microfiber bundles;
vi) dispersing the microfiber bundles prepared in step (iv) in a direction perpendicular to the longitudinal axis of the fibers, 6-12 times the original width of microfiber bundles using a dispersion unit to obtain a stretch responsive fibrillar matrix.
vii) maintaining the matrix prepared in step (vi) for 30-60 minutes in the fiber dispersion unit and size the matrix for biomedical use.

19. The method as claimed in claim 18, wherein the electrostatic spinning is carried out in electrospinning apparatus comprising Air compressor (A); Air blower with controller (B); Syringe pump (C); Electrospinning solution loaded syringe (D) with the needle; High voltage power supply (E); Electrostatically drawn polymer jet (F); Fiber bundle collector, which is a high-speed rotating mandrel (G).

20. The method as claimed in claim 18, wherein the electrostatic spinning in step (iv) is done at an applied voltage of at least 7.5 kV and the distance between the needle (D) to the mandrel (G) of electrospinning apparatus is at least 5 cm.

21. The method as claimed in claim 18, wherein the rate of the flow of homogeneous fibrous material solution or composite solution is at least 2.5 mL/hr.

22. The method as claimed in claim 18, wherein the viscosity of homogeneous fibrous material solution or composite solution is in the range of 1500 to 6000 Cp.

23. The method as claimed in claim 18, wherein the air flow volume near the mandrel (G) of the electrospinning apparatus is 60 CFM.
